(19) United States
(12) Patent Application Publication
(10) Pub. No.: US 2003/0023302 A1
(43) Pub. Date: Jan. 30, 2003

(54) Sewing cuff assembly for heart valves

(76) Inventors: Riyad Moe, Austin, TX (US); Tammi E. Klaco, Austin, TX (US); Matthew Allen Freund, Round Rock, TX (US); Edward Sarnowski, Cambridge, MA (US)

Correspondence Address: SULZER MEDICA USA INC., Suite 1600, 3 East Greenway Plaza, Houston, TX, 77046, US

(21) Appl. No.: 09/915,500

(22) Filed: Jul. 26, 2001

(51) Int. Cl. A61F 2/24

(52) U.S. Cl. 623/2.4

(57) ABSTRACT

The present invention provides a prosthetic valve and a sewing cuff assembly for attaching the prosthetic valve in a patient's heart. The sewing cuff generally includes at least one lock ring and sewing cuff material disposed at least partially around the lock ring. The valve body includes a peripheral groove for receiving the lock ring therein and securing the lock ring thereto. A stiffening ring may be coupled to the peripheral groove or otherwise provided on the valve body.

### BACKGROUND OF THE INVENTION

1. Field of the Invention

The present invention relates to the field of prosthetic heart valves and more particularly to a valve having a sewing cuff assembly that may be easily and securely coupled to the valve for implantation in an annulus of a human heart. In addition, the invention relates to a method for attaching a sewing cuff to a prosthetic heart valve.

2. Background of the Related Art

Prosthetic heart valves are used to replace diseased heart valves in humans. Prosthetic heart valves include mechanical heart valves, bioprosthetic valves using biological tissue, and polymer valves. The term “mechanical valve” as used herein refers to bi-leaflet heart valves comprising a valve orifice fabricated at least in part of a rigid, biologically compatible material such as pyrolytic carbon. Mechanical valves are rugged and long lasting in use, but use thereof in a patient requires anticoagulation drugs be taken by the patient in order to avoid clotting. The term “bioprosthetic valve” refers to a bi-leaflet or tri-leaflet heart valve comprising at least some biological components such as tissue or tissue components. The biological components of tissue valves are obtained from a donor animal (typically bovine or porcine), and the valve may comprise either biological materials alone or biological materials with man-made supports or stents. Tissue valves can be implanted without requiring the patient to use anticoagulation drugs but like natural valves, they tend to calcify and structurally degrade over time The term “polymeric valve” refers to a tri-leaflet or bi-leaflet heart valve comprising at least some elastomeric polymer components, including at least elastomeric polymer valve leaflets. Polymer valves are flexible by nature and therefore are closer to natural heart valves in use. Because they provide a more natural flow of blood, the polymer valves are less likely to cause coagulation that the mechanical valves. Because they can be made to exacting specifications, polymer valves are less prone to natural variations in size and wall thickness than tissue valves.

Conventional prosthetic heart valves, whether mechanical, bioprosthetic, or polymer valves, typically include an annular valve body comprising an orifice for blood flow through the valve. The valve body can be made of materials such as biocompatible pyrolitic carbon (mechanical valves), porcine or bovine pericardium tissue (bioprosthetic valves), or silicone or polyurethane (polymer valves). Leaflets are coupled to the annular valve body for movement between an open position and a closed position to allow or prevent blood flow through the orifice. Heart valves may include one, two or three leaflets. The leaflets can be made of pyrolytic carbon, treated tissue, or polymers.

Prosthetic heart valves are typically attached to a human heart with sutures via a sewing cuff. Sewing cuffs are generally attached to the periphery of the annular valve body to form a site for anchoring sutures to the annulus of the heart during implantation of the heart valve. Sewing cuffs are typically covered with a cloth material, such as polyester, and may also comprise a filler material such as Teflon felt or Dacron cloth. In mechanical valves, the sewing cuff may be coupled to a peripheral groove formed on the lower end of the valve body by circumferential cinch-like sutures. Alternatively, a ring may be disposed in a groove of the heart valve for stiffness and another ring or rings can be installed therearound with the sewing cuff material held between the rings.

More specifically, a prior art attachment assembly includes a ring disposed in a recess formed in a lower portion of a valve body. The ring increases the stiffness of the valve body and includes an annular groove formed around its outer perimeter. The sewing cuff assembly includes a pair of lock rings sewn into opposing ends of suture material. To assembly the sewing cuff to the valve body, the lock rings are snap fitted around the annular groove.

Any of the forgoing existing methods of coupling the sewing cuff to the valve body involve prolonged and repeated handling of the heart valve. Because attachment of the sewing cuff to the heart valve is a relatively labor-intensive and expensive part of valve fabrication, and because heart valves regardless of their type, are more susceptible to damage the more they are handled during assembly, packaging and storage, there is a need for a sewing ring that can be quickly and securely coupled to the valve body with minimal handling. Additionally, the number of parts making up conventional sewing cuff assemblies increases the complexity and the parts needed in inventory to complete an assembly.

Therefore, there is a need for a sewing cuff assembly which can be attached to a heart valve with a minimum of handling, and a method for rapidly and securely affixing a sewing cuff to a heart valve. There is also a need for a heart valve that has an attachment means for a sewing cuff using a minimal number of parts.

### SUMMARY OF THE INVENTION

The present invention generally provides a sewing cuff assembly and a heart valve to which the sewing cuff assembly is coupled. The invention further provides a method for attaching a sewing cuff assembly to a heart valve and attaching the assembled heart valve and sewing cuff assembly in a human heart.

In one aspect of the invention, a heart valve includes radially extending members extending outward from the lower surface of the valve body. The members are constructed and arranged to be received in mating apertures formed in a wall of a lock ring permitting sewing cuff material to be secured between the valve and the ring.

In another aspect, a heart valve includes a groove formed around a lower portion thereof and an inner ring disposed within the groove. The ring includes tabs extending radially outward therefrom for attachment to a lock ring that has mating formations and is disposable therearound. Fabric material of a sewing cuff is held between the rings.

In yet another aspect, the invention includes a mechanical heart valve with integrally formed tabs extending radially outward from a lower portion thereof. The tabs are constructed and arranged to receive a mating ring disposable around the valve body. Fabric material of a sewing cuff is held between the tabs and the ring.

In yet another aspect, the tabs are formed on a stent that is embedded in a polymer valve, the tabs extending outward from the surface of the valve.

### BRIEF DESCRIPTION OF THE DRAWINGS

So that the manner in which the above recited features, advantages and objects of the present invention are attained and can be understood in detail, a more particular description of the invention, briefly summarized above, may be had by reference to the embodiments thereof which are illustrated in the appended drawings.

It is to be noted, however, that the appended drawings illustrate only typical embodiments of this invention and are therefore not to be considered limiting of its scope, for the invention may admit to other equally effective embodiments.

FIG. 1A is a perspective view of a heart valve having an embedded s therein with outwardly extending tabs extending radially outward from the lower valve body.

FIG. 1B is a perspective view, partially in section, of a sewing cuff assembly with a ring disposed in the inner portion thereof.

FIG. 2A is a cross-sectional view of a valve body having a sewing cuff retained on the valve by the inner and outer rings.

FIG. 2B is a perspective view showing the inner and outer rings of the assembly of FIG. 2A.

FIG. 3 is a perspective view of an implanted heart valve attached to human tissue with sutures.

### DETAILED DESCRIPTION OF THE PREFERRED EMBODIMENT

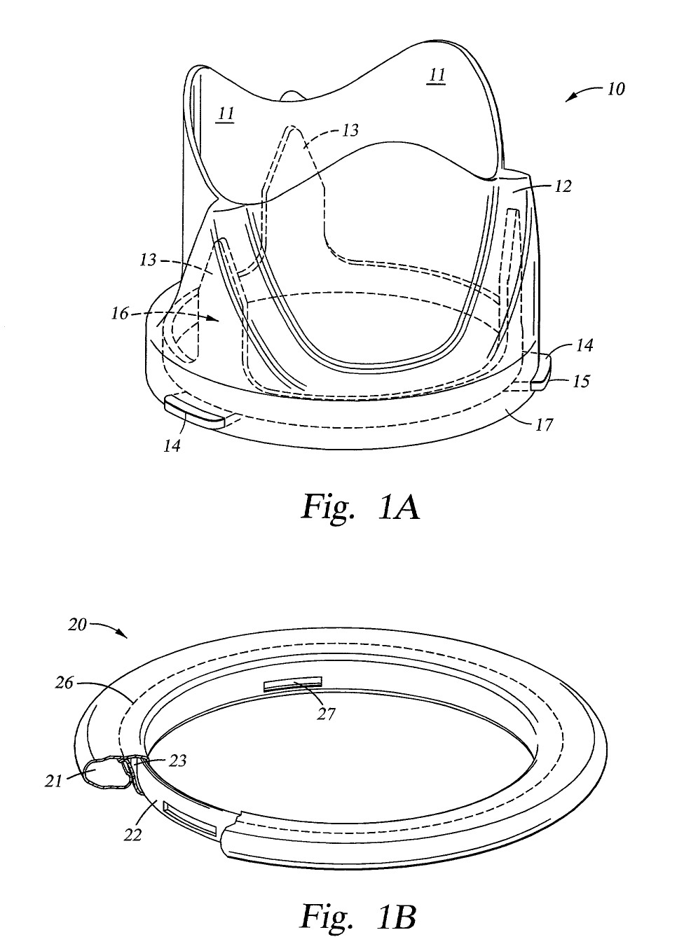

FIG. 1A is a perspective view of a tri-leaflet prosthetic heart valve 10 having an annular valve body 12 and three flexible leaflets 11 made of a biocompatible polymer such as silicone or polyurethane. The valve body 12 includes a base portion having a periphery 17 defining an outer diameter. A stent 16, made of metal or plastic, is embedded or otherwise disposed in the valve body 12 for reinforcement. In FIG. 1A, the stent 16 includes upright portions 13 designed to provide support to the upper areas of the annular valve body 12. The stent also includes at least two outwardly extending members 14, extending outward from the periphery 17 of the valve body to increase the overall outer circumference of the valve body. In the embodiment shown, the members 14 are formed as tabs having an outer surface 15 following the curvature of the periphery of the valve body 12.

FIG. 1B is a perspective view, partially in section, of a sewing cuff 20 having an outer ring 22 disposed in an inner portion 23 thereof. The sewing cuff 20 is designed to be coupled to the valve body 12 at the periphery 17 and allows a surgeon to suture the prosthetic heart valve 10 to a natural heart. The sewing cuff 20 includes an outer portion 21 that may be hollow and is typically for attachment to heart tissue. A filler material (not shown) can optionally be placed within outer portion 21 in the hollow space to provide reinforcement and rigidity and facilitate suturing. Filler material can include Teflon felt or Dacron, for example. The sewing cuff 20, including both the outer portion 21 and any filler material, helps prevent perivalvular leakage around the implanted valve 10 when the valve is closed. As illustrated in FIG. 1B, the inner portion 23 is formed when a free end of the sewing material is folded back on itself and sewn along a line 26 to form a tubular shape housing the outer ring 22. Likewise, the outer portion 21 is formed when a portion of the sewing material is formed into a tubular shape and sewn along line 26.

Still referring to FIG. 1B, the outer ring 22 includes at least two apertures 27 formed in a wall thereof. The apertures are constructed and arranged to receive the tabs 14 extending from the periphery of the valve body 12 when the outer ring 22 is snap-fitted over the valve body at periphery 17. In FIG. 1B, the apertures 27 are shown extending completely though the wall of the outer ring 22. However, the apertures could be formed as indentations in the inner surface of the ring 22 and still receive the tabs 14 of the valve body 12 in a locking relationship. The outer ring 22 preferably has an inner circumference slightly smaller than the circumference of the valve body, measured around the outer surface of the tabs 14. Additionally, the outer ring 22 is constructed of a geometry and/or material providing adequate flexibility to permit the ring 22 to be extended over the tabs 14 during assembly. Alternatively, the stent 16 and/or valve body 12 are constructed with adequate flexibility to permit a less flexible outer ring to be disposed therearound. With the outer ring 22 disposed over the periphery of the valve body and the tabs extending into the apertures, an inside portion of the suture material making up the inner portion 23 of the sewing cuff 20 is securely retained between the outer ring 22 and the valve body periphery 17. After assembly, the valve and sewing cuff assembly is ready for implantation.

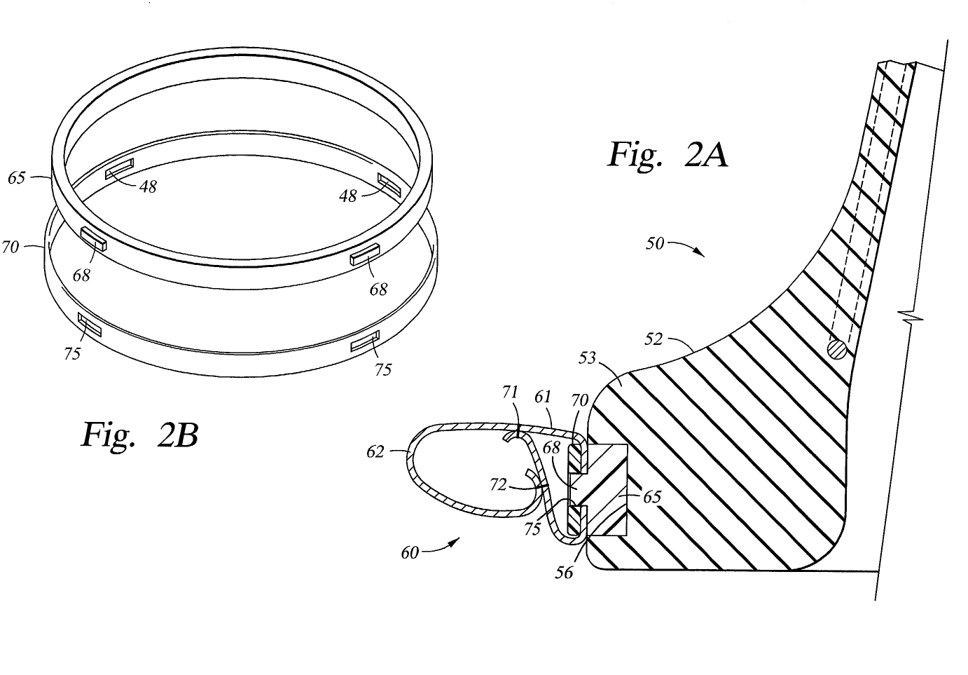

FIG. 2A is a cross-sectional view of a heart valve 50 illustrating another embodiment of a sewing cuff assembly 60 of the present invention. The valve body 52 includes a base portion 53 with a circumferential groove 56 formed on the periphery therearound. An inner ring 65 is disposed in the groove 56 and coupled to or otherwise secured to the heart valve by molding, press fitting the component into place, or other fabrication techniques known in the art, e.g. adhesives. In a polymer valve, the inner ring 65 can provide support to stiffen the valve body and facilitate the installation of a sewing cuff. On the outer surface of inner ring 65 are at least two tabs 68 that effectively enlarges the circumference of the base portion 53 of the valve body 52 when measured at the outer surface of the tabs 68.

An outer ring 70, shown in section in FIG. 2A and in perspective in FIG. 2B, preferably has an inner circumference that is slightly less than the circumference as measured around the outer surface of the tabs 68. This ensures that the outer ring 70 is held securely around the inner ring 65 after it is snap-fitted onto the inner ring 65. Apertures 75 are formed in a wall of the outer ring 70 to mate with the tabs 68 of inner ring 65. The inner ring 65 can be made of biocompatible material such as cobalt chromium or titanium.

The sewing cuff assembly 60 shown in FIGS. 2A and 2B includes an inner portion 61 and an outer portion 62. The fabric making up the inner and outer portions can be polyester, Teflon felt or Dacron. As shown in FIG. 2A, the inner and outer portions of the sewing cuff are formed by stitching. The inner portion 61 is formed by sewing a free end of the sewing cuff fabric at a line 71, with the outer ring enclosed thereon. When the sewing cuff is assembled to the valve body 52 as shown in FIG. 2A, at least some portion of the fabric of the inner portion 61 is retained between the inner 65 and outer 70 rings. Other methods of coupling the suture material to the ring, such as stapling, will be apparent to persons of skill in the art.

The outer portion of the sewing cuff is likewise formed by sewing a second free end of the fabric material along a line 72. The outer portion is thereafter used to sew the sewing ring to a human heart during implantation of the valve 50. As with the embodiment of FIG. 1A and 1B, the outer portion of the sewing ring can include a filler material of Teflon felt, Dacron cloth or gel material.

The sewing cuff assembly according to FIGS. 2A and 2B can be assembled and coupled to a heart valve as follows. The outer ring 70 is positioned inside the inner portion 61 of the sewing cuff made of a material like polyester and sutured, stapled or otherwise secured in place. An optional filler material can be enclosed in an outer portion 62 of the sewing cuff 60. An inner ring 65, such as a ring illustrated in FIGS. 2A and 2B having tabs 68 formed on the outer surface thereof, is coupled to groove 56 in the periphery of heart valve 50. The outer ring 70 is then snapped-fitted around the inner ring. Preferably, the inner ring 65 is made of a material such as titanium, which enables the outer ring 70 to be positioned around the inner ring in much the same way as a clincher bicycle tire is fitted onto a bicycle rim.

In this manner, the outer ring 70 is slightly distorted as it fits over the tabs of the inner ring 65.

As shown in the embodiment of FIG. 2A, the material making up the inner portion 61 of the sewing cuff is preferably folded back and stitched at a location 71 adjacent the outer ring 70. Additionally, the material making up the outer portion 62 of the sewing cuff is also folded back and sewn at a location 72. This configuration of the suture material provides a sufficient amount and thickness of suture material to enable the suture material to be reliably secured in a heart and to fill any space between the valve body and a heart annulus in which the valve is positioned. As with the embodiment of FIGS. 1A-1B, filler material could also be used to fill outer portion 62.

The assembled heart valve can then be sutured into the annulus of a patient's heart after the native valve is removed. This configuration of a sewing cuff assembly enables rapid fabrication of a heart valve with minimal contact with the valve body. In addition, this configuration eliminates the need for sutures or pins to secure the sewing cuff to the valve body.

Because the embodiment of FIGS. 2A-2B utilizes a single ring around the valve body, the portion of the valve supporting the ring can have a lower vertical height than a conventional, two lock ring embodiment. The vertical height is a consideration in the application or position the valve is to be inserted. For example, in the aortic position, surgeons prefer to use a smaller valve. Therefore, a valve having a reduced or minimized height may be provided by a single ring embodiment.

The shape, configuration and materials used in the embodiments of FIGS. 2A and 2B are the same or similar to those described above in reference to the embodiment of FIG. 1A and 1B.

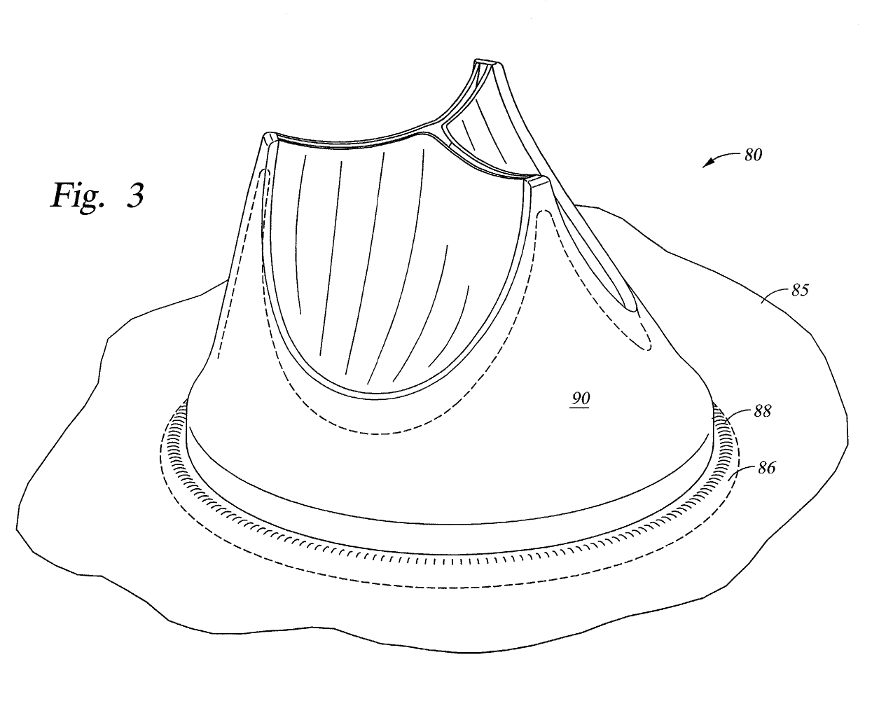

FIG. 3 is a perspective view of a prosthetic heart valve 80 attached to natural heart tissue, e.g., annulus 85 of a patient. The heart valve 80 is sewn into place by suturing the annulus tissue to the sewing cuff 86 by stitches 88. Rings (not shown) secure the sewing cuff to the valve body 90 and the assembly provides attachment of the prosthetic heart valve in a patient's heart.

While the embodiments shown illustrate the tabs formed on either a stent or a ring disposed in the valve body, it will be understood that the tabs could be formed directly on the valve body, in the case of a material having adequate rigidity to support the tabs. Alternately, tabs could be formed on an inner surface of an outer ring and the inner ring (or the valve body itself) could have mating apertures formed therein for locking attachment to the inwardly facing tabs of the outer ring.

While the foregoing is directed to the preferred embodiment of the present invention, other and further embodiments of the invention may be devised without departing from the basic scope thereof, and the scope thereof is determined by the claims that follow.

What is claimed is:

1. A heart valve, comprising:
(a) a valve body having an outer circumferential portion having at least two radially outwardly extending members;
(b) a sewing cuff assembly comprising:
(i) at least one outer ring, the outer ring including at least two formations for receiving the at least two members of the valve body; and
(ii) a material disposed at least partially around and secured to the outer ring.

2. The heart valve of claim 1, wherein the at least two formations on the outer ring are apertures formed in a wall of the ring, the apertures constructed and arranged to receive the at least two members of the valve body when the outer ring is disposed around the valve body.

3. The heart valve of claim 2, wherein the at least two members are tabs, the tabs having an outer surface enlarging the circumference of the valve body.

4. The heart valve of claim 3, wherein the tabs are formed on a stent, the stent at least partially embedded in the valve body.

5. The heart valve of claim 4, wherein the inner circumference of the outer ring is smaller than the outer circumference of the valve body as measured around the outer surface of the tabs.

6. The heart valve of claim 5, wherein the outer ring is disposable on the valve body, the tabs of the valve body lockable in the apertures of the outer ring.

7. The heart valve of claim 6, wherein the material disposed at least partially around the outer ring is retained between the valve body and the outer ring.

8. The heart valve of claim 7, wherein the material forms an inner tubular portion and the outer ring is disposable within the inner tubular portion.

9. The heart valve of claim 8, wherein the material additionally forms an outer tubular portion, the outer tubular portion constructed and arranged to facilitate attachment of the heart valve to a human heart during implantation of the heart valve.

10. The heart valve of claim 9, wherein the outer tubular portion further comprises a filler material disposed therein.

11. The heart valve of claim 1, wherein the circumferential portion includes a groove formed therein and further includes an inner ring disposed in the groove.

12. The heart valve of claim 11, wherein the at least two radially outwardly extending members are formed on the inner ring.

13. The heart valve of claim 12, wherein the inner ring is a stiffening ring providing support to the valve body.

14. The heart valve of claim 1, wherein the valve body comprises a material selected from the group of polyurethane, silicone or combinations thereof.

15. The heart valve of claim 3, wherein the tabs are integrally formed on the valve body.

16. The heart valve of claim 13, wherein the stiffening ring is discontinuous around the perimeter of the valve body.

17. The heart valve of claim 1, wherein the material is selected from the group of polyester, Teflon felt, and Dacron.

18. A method of assembling a heart valve assembly, the method comprising:
(a) sewing an outer ring having formations formed in a wall thereof in a sewing cuff;
(b) aligning the formations of the outer ring with mating members formed on the valve; and
(c) fitting the outer ring over the valve whereby the mating members are retained in the formations, thereby retaining the sewing cuff fabric between the outer ring and the valve.

19. A heart valve, comprising:
(a) a valve body comprising a flow orifice and an outer periphery and at least two apertures within the outer periphery;
(b) a sewing cuff assembly comprising:
(i) an outer ring with at least two members extending radially inward therefrom for engaging the at least two apertures of the valve body; and
(ii) a material disposed at least partially around and coupled to an outer ring.
